(19) United States
(12) Patent Application Publication
(10) Pub. No.: US 2007/0123958 A1
(43) Pub. Date: May 31, 2007

(54) APPARATUS FOR TREATING AIRWAYS IN THE LUNG

(75) Inventor: Michael D. LAUFER, Menlo Park, CA (US)

Correspondence Address: ASTHMATX, INC.;c/o LEVINE BAGADE HAN, LLP, 2483 EAST BAYSHORE ROAD, SUITE 100, PALO ALTO, CA, 94303, US

(73) Assignee: ASTHMATX, INC., Mountain View, CA (US)

(21) Appl. No.: 11/562,910

(22) Filed: Nov. 22, 2006

Related U.S. Application Data
(63) Continuation-in-part of application No. 10/640,967, filed on Aug. 13, 2003.
Application No. 10/640,967 is a continuation of application No. 09/535,856, filed on Mar. 27, 2000, now Pat. No. 6,634,363.
Application No. 09/535,856 is a continuation-in-part of application No. 09/296,040, filed on Apr. 21, 1999, now Pat. No. 6,411,852.
Application No. 09/296,040 is a continuation-in-part of application No. 09/095,323, filed on Jun. 10, 1998.

(51) Int. Cl. A61N 5/06

(52) U.S. Cl. 607/93

(57) ABSTRACT

A device and method for treating bodily conduits involves the application of energy to the smooth muscle tissue of the conduit walls to reduce the bulk of smooth muscle tissue and mucus glands. The irradiation treatment of the smooth muscle tissue causes a reduction in the amount of smooth muscle tissue over time which increases the inner diameter of the body conduit for improved fluid flow and prevents smooth muscle spasms. The treatment is particularly useful in the lungs for treatment of asthma to prevent bronchospasms, increase the airway diameter for improved air exchange, and reduce mucus secretions in the lungs.

This is a continuation-in-part application of U.S. application Ser. No. 10/640,967, filed Aug. 13, 2003 which is a continuation of U.S. application Ser. No. 09/535,856, filed Mar. 27, 2000, now U.S. Pat. No. 6,634,363 which is a continuation-in-part of U.S. application Ser. No. 09/296,040, filed Apr. 21, 1999, now U.S. Pat. No. 6,411,852 which is a continuation-in-part of U.S. application Ser. No. 09/095,323, filed Jun. 10, 1998. All the above applications are incorporated by reference herein in their entirety.

### BACKGROUND OF THE INVENTION

1. Field of the Invention

Generally, the present invention relates to medical devices and methods. In particular, the invention relates to a method of treating a lung having at least one symptom of reversible obstructive pulmonary disease, and more particularly, the invention relates to advancing a treatment device into the lung and treating the lung with the device to at least reduce the ability of the lung to produce at least one of the symptoms of reversible obstructive pulmonary disease. The invention includes additional steps that reduce the ability of the lung to produce at least one of the symptoms of reversible obstructive pulmonary disease and to reduce the resistance to the flow of air through a lung. The invention also relates to a method and apparatus for treating smooth muscle in the walls of body conduits, and more particularly, the invention relates to a method for treating medical conditions by reducing the bulk of smooth muscle surrounding a body conduit with radiant energy treatment of the smooth muscle.

2. Brief Description of the Related Art

Reversible obstructive pulmonary disease includes asthma and reversible aspects of chronic obstructive pulmonary disease (COPD). Asthma is a disease in which (i) bronchoconstriction, (ii) excessive mucus production, and (iii) inflammation and swelling of airways occur, causing widespread but variable airflow obstruction thereby making it difficult for the asthma sufferer to breathe. Asthma is a chronic disorder, primarily characterized by persistent airway inflammation. However, asthma is further characterized by acute episodes of additional airway narrowing via contraction of hyper-responsive airway smooth muscle.

The reversible aspects of COPD generally describe excessive mucus production in the bronchial tree. Usually, there is a general increase in bulk (hypertrophy) of the large bronchi and chronic inflammatory changes in the small airways. Excessive amounts of mucus are found in the airways and semisolid plugs of mucus may occlude some small bronchi. Also, the small airways are narrowed and show inflammatory changes. The reversible aspects of COPD include partial airway occlusion by excess secretions, and airway narrowing secondary to smooth muscle contraction, bronchial wall edema and inflation of the airways

In asthma, chronic inflammatory processes in the airway play a central role in increasing the resistance to airflow within the lungs. Many cells and cellular elements are involved in the inflammatory process, particularly mast cells, eosinophils T lymphocytes, neutrophils, epithelial cells, and even airway smooth muscle itself. The reactions of these cells result in an associated increase in the existing sensitivity and hyper-responsiveness of the airway smooth muscle cells that line the airways to the particular stimuli involved.

The chronic nature of asthma can also lead to remodeling of the airway wall (i.e., structural changes such as thickening or edema) which can further affect the function of the airway wall and influence airway hyper-responsiveness. Other physiologic changes associated with asthma include excess mucus production, and if the asthma is severe, mucus plugging, as well as ongoing epithelial denudation and repair. Epithelial denudation exposes the underlying tissue to substances that would not normally come in contact with them, further reinforcing the cycle of cellular damage and inflammatory response.

In susceptible individuals, asthma symptoms include recurrent episodes of shortness of breath (dyspnea), wheezing, chest tightness, and cough. Currently, asthma is managed by a combination of stimulus avoidance and pharmacology.

Stimulus avoidance is accomplished via systematic identification and minimization of contact with each type of stimuli. It may, however, be impractical and not always helpful to avoid all potential stimuli.

Asthma is managed pharmacologically by: (1) long term control through use of anti-inflammatories and long-acting bronchodilators and (2) short term management of acute exacerbations through use of short-acting bronchodilators. Both of these approaches require repeated and regular use of the prescribed drugs. High doses of corticosteroid anti-inflammatory drugs can have serious side effects that require careful management. In addition, some patients are resistant to steroid treatment. The difficulty involved in patient compliance with pharmacologic management and the difficulty of avoiding stimulus that triggers asthma are common barriers to successful asthma management.

Asthma is a serious disease with growing numbers of sufferers. Current management techniques are neither completely successful nor free from side effects.

Accordingly, it would be desirable to provide an asthma treatment which improves airflow without the need for patient compliance.

In addition to the airways of the lungs, other body conduits such as the esophagus, ureter, urethra, and coronary arteries, are also subject to periodic reversible spasms that produce obstruction to flow.

### SUMMARY OF THE INVENTION

The present invention relates to a device and method for treating bodily conduits by application of radiant energy to the smooth muscle tissue of the conduit walls to prevent the smooth muscle tissue from replicating. The treatment of the smooth muscle tissue causes a reduction in the amount of smooth muscle tissue over time which increases the inner diameter of the body conduit and prevents smooth muscle spasms.

In accordance with one aspect of the present invention, an apparatus for the treatment of body conduits includes an elongate body configured to be inserted into a body conduit, the elongate body having a proximal end and a distal end, and a source of energy for emitting energy from the elongate body in an intensity which, when applied to walls of the body conduit causes a change in smooth muscle tissue which prevents the smooth muscle tissue from replicating.

In accordance with another aspect of the present invention, an apparatus for the treatment of walls of airways in a patient's lungs includes an elongate body configured to be inserted into the airways of a patient's lungs, the device having a proximal end and a distal end, and a source of energy for emitting energy from the distal end of the elongate body in an intensity which, when applied to the walls of the airway causes a change in smooth muscle tissue which prevents the smooth muscle tissue from replicating.

When the source of energy is a light source the apparatus further includes a light transmitting fiber extending from the proximal end to the distal end of the elongate body for transmitting light from the light source into the patient's lungs, a connector on the distal end of the elongate body for connecting the elongate body to the source of light, and a light directing member positioned at a distal end of the elongate device for diffusing or redirecting the light from the light transmitting fiber in a substantially radial pattern from the distal end of the elongate device.

In accordance with an additional aspect of the present invention, a method of treating asthma to control bronchospasms includes irradiating the walls of an airway in a lung in a wavelength and intensity which causes a change in smooth muscle tissue cells and prevents the smooth muscle tissue cells from replicating, and controlling bronchospasms by reduction or elimination of smooth muscle tissue.

In accordance with a further aspect of the invention, a method of treating respiratory conditions to control mucus plugging includes irradiating the walls of an airway in a lung in a wavelength and intensity which causes a change in mucus gland cells and prevents the mucus gland cells from replicating, and preventing mucus plugging by reduction or elimination of mucus glands.

In accordance with another aspect of the present invention, a method of treating an esophagus, an ureter, or an urethra to control spasms includes irradiating the walls of a conduit to cause a change in smooth muscle cells and prevent the smooth muscle cells from replicating.

The present invention provides advantages of a treatment for asthma or other enlargement or spasm of the smooth muscle by irradiation. The treatment enlarges airways, reduces or eliminates mucus plugging, and reduces or eliminates bronchospasm.

The present invention relates to methods for treating a lung, preferably having at least one symptom of reversible obstructive pulmonary disease, comprising the steps of advancing a treatment device into the lung and treating the lung with the device to at least reduce the ability of the lung to produce at least one symptom of reversible obstructive pulmonary disease and to decrease the resistance to the flow of air through the lung.

A variation of the invention includes the method described above further comprising the step of locating one or more treatment sites within an airway of the lung, selecting at least one of the treatment sites and treating at least one of the treatment sites selected in the selecting step. The invention may further include performing the steps while the lung is experiencing at least one symptom of either natural or artificially induced reversible obstructive pulmonary disease.

A further variation of the invention includes the method described above and further includes the steps of testing the lung for at least one pre-treatment pulmonary function value prior to the treating step, and re-testing the lung for at least one post-treatment pulmonary function value subsequent to the treating step.

A further variation of the invention includes the method described above further comprising identifying treatment sites within the airway being highly susceptible to either airway inflammation, airway constriction, excessive mucus secretion, or any other symptom of reversible obstructive pulmonary disease.

Another variation of the invention includes the method described above and the additional step of stimulating the lung to produce at least one artificially induced symptom of reversible obstructive pulmonary disease. The invention may further comprise the step of evaluating the results of the stimulating step.

Another variation of the invention includes the method described above where treating at least airway tissue within the lung further comprises the step of determining the effect of the treatment by visually observing the airway for blanching of airway tissue.

Another variation of the invention includes the method described above where treating at least airway tissue at a treatment site within the lung further comprises the step of monitoring electrical impedance of tissue at one or more points.

Another variation of the invention includes the method described above where treating the lung includes sub-mucosal treatment of at least airway tissue in the lung.

Another variation of the invention includes the method described above where the treating step includes treating the lung by depositing a radioactive substance in at least one treatment site within the lung.

Another variation of the invention include the method described above further including the step of scraping tissue from a wall of an airway within the lung prior to the treating step. The invention may further comprise depositing a substance on the scraped wall of the airway.

Another variation of the invention includes the method described above where the treating step uses a modality selected from the group consisting of mechanical, chemical, radio frequency, radioactive energy, heat, and ultrasound.

Another variation of the invention includes the method described above further comprising pre-treating the lung to at least reduce the ability of the lung to produce at least one symptom of reversible obstructive pulmonary disease prior to the treating step, where at least one parameter of the pre-treating step is lesser than at least one parameter of the treating step.

Another variation of the invention comprises the method described above where the treating step includes separating the treating step into stages to reduce the healing load on the lung. The separating step may comprise treating different regions of the lung at different times or dividing the number of treatment sites into a plurality of groups of treatment sites and treating each group at a different time.

Another variation of the invention includes the method described above further comprising sensing movement of the lung and repositioning the treatment device in response to said sensing step.

Another variation of the invention includes the method described above further comprising reducing the temperature of lung tissue adjacent to a treatment site.

Another variation of the invention includes the method described above further comprising the step of providing drug therapy, exercise therapy, respiratory therapy, and/or education on disease management techniques to further reduce the effects of reversible obstructive pulmonary disease.

The invention further includes the method for reversing a treatment to reduce the ability of the lung to produce at least one symptom of reversible obstructive pulmonary disease comprising the step of stimulating re-growth of smooth muscle tissue in the lung.

The invention further includes the method of evaluating an individual having reversible obstructive pulmonary disease as a candidate for a procedure to reduce the ability of the individual's lung to produce at least one reversible obstructive pulmonary disease symptom by treating an airway within the lung of the individual, the method comprising the steps of assessing the pulmonary condition of the individual, comparing the pulmonary condition to a corresponding predetermined state; and evaluating the individual based upon the comparing step. The method may additionally comprise the steps of performing pulmonary function tests on the individual to obtain at least one pulmonary function value, comparing the at least one pulmonary function value to a corresponding predetermined pulmonary function value, and evaluating the individual based upon the comparing step.

The invention further comprises a method of evaluating the effectiveness of a procedure to reduce the ability of lung to produce at least one symptom of reversible obstructive pulmonary disease previously performed on an individual having reversible obstructive pulmonary disease, the method comprising the steps of assessing the pulmonary condition of the individual, comparing the pulmonary condition to a corresponding predetermined state; and evaluating the effectiveness of the procedure based upon the comparing step. The method may additionally comprise the steps of performing pulmonary function tests on the individual to obtain at least one pulmonary function value, treating the lung to at least reduce the ability of the lung to produce at least one symptom of reversible obstructive pulmonary disease, performing post-procedure pulmonary function tests on the individual to obtain at least one post-procedure pulmonary function value; and comparing the pulmonary function value with the post-procedure pulmonary function value to determine the effect of the treating step.

### BRIEF DESCRIPTION OF THE DRAWING

The invention will now be described in greater detail with reference to the various embodiments illustrated in the accompanying drawings:

FIG. 1. is a cross sectional view of an airway in a healthy lung.

FIG. 2. shows a section through a bronchiole having an airway diameter smaller than that shown in FIG. 1.

FIG. 3 illustrates the airway of FIG. 1 in which the smooth muscle 14 has hypertrophied and increased in thickness causing reduction of the airway diameter.

FIG. 4 is a schematic side view of the lungs being treated with a treatment device 38 as described herein.

FIG. 5 is a side cross sectional view of a body conduit and another apparatus for treating the body conduit;

FIG. 6 is a schematic side view of lungs being treated with a treatment device; and

FIGS. 7-12 are side cross sectional views of distal ends of additional first embodiment of treatment devices according to the present invention.

### DETAILED DESCRIPTION

The invention relates to methods for improving airflow through the airways of a lung having reversible obstructive pulmonary disease. It is intended that the invention is applicable to any aspect of reversible obstructive pulmonary disease, including but not limited to asthma. One way of improving airflow is to decrease the resistance to airflow within the lungs. There are several approaches to reducing this resistance, including but not limited to reducing the ability of the airway to contract, increasing the airway diameter, reducing the inflammation of airway tissues, and/or reducing the amount of mucus plugging of the airway. The present invention includes advancing a treatment device into the lung and treating the lung to at least reduce the ability of the lung to produce at least one symptom of reversible obstructive pulmonary disease. The following is a brief discussion of some causes of increased resistance to airflow within the lungs and the inventive treatment of the invention described herein. As such, the following discussion is not intended to limit the aspects or objective of the inventive method as the inventive method may cause physiological changes not described below but such changes still contributing to reducing or eliminating at least one of the symptoms of reversible obstructive pulmonary disease.

Reducing the Ability of the Airway to Contract

The inventive treatment reduces the ability of the airways to narrow or to reduce in diameter due to airway smooth muscle contraction. The inventive treatment uses a modality of treatments including, but not limited to the following: chemical, radio frequency, radioactivity, heat, ultrasound, radiant, laser, microwave, or mechanical energy (such as in the form of cutting, punching, abrading, rubbing, or dilating). This treatment reduces the ability of the smooth muscle to contract thereby lessening the severity of an asthma attack. The reduction in the ability of the smooth muscle to contract may be achieved by treating the smooth muscle itself or by treating other tissues which in turn influence smooth muscle contraction or the response of the airway to the smooth muscle contraction. Treatment may also reduce airway responsiveness or the tendency of the airway to narrow or to constrict in response to a stimulus.

The amount of smooth muscle surrounding the airway can be reduced by exposing the smooth muscle to energy which either kills the muscle cells or prevents these cells from replicating. The reduction in smooth muscle reduces the ability of the smooth muscle to contract and to narrow the airway during a spasm. The reduction in smooth muscle and surrounding tissue has the added potential benefit of increasing the caliber or diameter of the airways, this benefit reduces the resistance to airflow through the airways. In addition to the use of debulking smooth muscle tissue to open up the airways, the device used in the present invention may also eliminate smooth muscle altogether by damaging or destroying the muscle. The elimination of the smooth muscle prevents the contraction or spasms of hyper-reactive airways of a patient having reversible obstructive pulmonary disease. By doing so, the elimination of the smooth muscle may reduce some symptoms of reversible obstructive pulmonary disease.

The ability of the airway to contract can also be altered by treatment of the smooth muscle in particular patterns. The smooth muscle is arranged around the airways in a generally helical pattern with pitch angles ranging from about −38 to about +38 degrees. Thus, the treatment of the smooth muscle in appropriate patterns interrupts or cuts through the helical pattern of the smooth muscle at a proper pitch and prevents the airway from constricting. This procedure of patterned treatment application eliminates contraction of the airways without completely eradicating smooth muscle and other airway tissue. A pattern for treatment may be chosen from a variety of patterns including longitudinal or axial stripes, circumferential bands, helical stripes, and the like as well as spot patterns having rectangular, elliptical, circular or other shapes. The size, number, and spacing of the treatment bands, stripes, or spots are chosen to provide a desired clinical effect of reduced airway responsiveness while limiting insult to the airway to a clinically acceptable level.

The patterned treatment of the tissues surrounding the airways with energy provides various advantages. The careful selection of the portion of the airway to be treated allows desired results to be achieved while reducing the total healing load. Patterned treatment can also achieve desired results with decreased morbidity, preservation of epithelium, and preservation of a continuous or near continuous ciliated inner surface of the airway for mucociliary clearance. The pattern of treatment may also be chosen to achieve desired results while limiting total treatment area and/or the number of airways treated, thereby improving speed and ease of treatment.

Application of energy to the tissue surrounding the airways may also cause the DNA of the cells to become cross linked. The treated cells with cross linked DNA are incapable of replicating. Accordingly, over time, as the smooth muscle cells die, the total thickness of smooth muscle decreases because of the inability of the cells to replicate. The programmed cell death causing a reduction in the volume of tissue is called apoptosis. This treatment does not cause an immediate effect but causes shrinking of the smooth muscle and opening of the airway over time and substantially prevents re-growth. The application of energy to the walls of the airway may also be used to cause a cross linking of the DNA of the mucus gland cells thereby preventing them from replicating and reducing excess mucus plugging or production over time.

The ability of the airways to contract may also be reduced by altering mechanical properties of the airway wall, such as by increasing stiffness of the wall or by increasing parenchymal tethering of the airway wall. Both of these methods increase the strength of the airway wall and further oppose contraction and narrowing of the airway.

There are several ways to increase the stiffness of the airway wall. One way to increase stiffness is to induce fibrosis or a wound healing response by causing trauma to the airway wall. The trauma can be caused by delivery of therapeutic energy to the tissue in the airway wall, by mechanical insult to the tissue, or by chemically affecting the tissue. The energy is preferably delivered in such a way that it minimizes or limits the intra-luminal thickening that may occur.

Another way to increase the effective stiffness of the airway wall is to alter the submucosal folding of the airway upon narrowing. The mucosal layer includes the epithelium, its basement membrane, and the lamina propria, a subepithelial collagen layer. The submucosal layer may also play a role in airway folding. As an airway narrows, its perimeter remains relatively constant, with the mucosal layer folding upon itself. As the airway narrows further, the mucosal folds mechanically interfere with each other, effectively stiffening the airway. In asthmatic patients, the number of folds is fewer and the size of the folds is larger, and thus, the airway is free to narrow with less mechanical interference of mucosal folds than in a healthy patient. Thus, asthmatic patients have a decrease in airway stiffness and the airways have less resistance to narrowing.

The mucosal folding in asthmatic patients can be improved by treatment of the airway in a manner which encourages folding. Preferably, a treatment will increase the number of folds and/or decrease the size of the folds in the mucosal layer. For example, treatment of the airway wall in a pattern such as longitudinal stripes can encourage greater number of smaller mucosal folds and increase airway stiffness.

The mucosal folding can also be increased by encouraging a greater number of smaller folds by reducing the thickness of the mucosa and/or submucosal layer. The decreased thickness of the mucosa or submucosa may be achieved by application of energy which either reduces the number of cells in the mucosa or submucosal layer or which prevents replication of the cells in the mucosa or submucosal layer. A thinner mucosa or submucosal layer will have an increased tendency to fold and increased mechanical stiffening caused by the folds.

Another way to reduce the ability of the airways to contract is to improve parenchymal tethering. The parenchyma surrounds airways and includes the alveolus and tissue connected to and surrounding the outer portion of the airway wall. The parenchyma includes the alveolus and tissue connected to and surrounding the cartilage that supports the larger airways. In a healthy patient, the parenchyma provides a tissue network which connects to and helps to support the airway. Edema or accumulation of fluid in lung tissue in patients with asthma or COPD is believed to decouple the airway from the parenchyma reducing the restraining force of the parenchyma which opposes airway constriction. Energy can be used to treat the parenchyma to reduce edema and/or improve parenchymal tethering.

In addition, the applied energy may be used to improve connection between the airway smooth muscle and submucosal layer to the surrounding cartilage, and to encourage wound healing, collagen deposition, and/or fibrosis in the tissue surrounding the airway to help support the airway and prevent airway contraction.

Increasing the Airway Diameter

Hypertrophy of smooth muscle, chronic inflammation of airway tissues, and general thickening of all parts of the airway wall can reduce the airway diameter in patients with reversible obstructive pulmonary disease. Increasing the overall airway diameter using a variety of techniques can improve the passage of air through the airways. Application of energy to the airway smooth muscle of an asthmatic patient can debulk or reduce the volume of smooth muscle. This reduced volume of smooth muscle increases the airway diameter for improved air exchange.

Reducing inflammation and edema of the tissue surrounding the airway can also increase the diameter of an airway. Inflammation and edema (accumulation of fluid) of the airway are chronic features of asthma. The inflammation and edema can be reduced by application of energy to stimulate wound healing and regenerate normal tissue. Healing of the epithelium or sections of the epithelium experiencing ongoing denudation and renewal allows regeneration of healthy epithelium with less associated airway inflammation. The less inflamed airway has an increased airway diameter both at a resting state and in constriction. The wound healing can also deposit collagen which improves parenchymal tethering.

Inflammatory mediators released by tissue in the airway wall may serve as a stimulus for airway smooth muscle contraction. Therapy that reduces the production and release of inflammatory mediator can reduce smooth muscle contraction, inflammation of the airways, and edema. Examples of inflammatory mediators are cytokines, chemokines, and histamine. The tissues which produce and release inflammatory mediators include airway smooth muscle, epithelium, and mast cells. Treatment of these structures with energy can reduce the ability of the airway structures to produce or release inflammatory mediators. The reduction in released inflammatory mediators will reduce chronic inflammation, thereby increasing the airway inner diameter, and may also reduce hyper-responsiveness of the airway smooth muscle.

A further process for increasing the airway diameter is by denervation. A resting tone of smooth muscle is nerve regulated by release of catecholamines. Thus, by damaging or eliminating nerve tissue in the airways the resting tone of the smooth muscle is reduced, and the airway diameter is increased. Resting tone may also be reduced by directly affecting the ability of smooth muscle tissue to contract.

Reducing Plugging of the Airway

Excess mucus production and mucus plugging are common problems during both acute asthma exacerbation and in chronic asthma management. Excess mucus in the airways increases the resistance to airflow through the airways by physically blocking all or part of the airway. Excess mucus may also contribute to increased numbers of leukocytes found in airways of asthmatic patients by trapping leukocytes. Thus, excess mucus can increase chronic inflammation of the airways.

One type of asthma therapy involves treatment of the airways with energy to target and reduce the amount of mucus producing cells and glands and to reduce the effectiveness of the remaining mucus producing cells and glands. The treatment can eliminate all or a portion of the mucus producing cells and glands, can prevent the cells from replicating or can inhibit their ability to secrete mucus. This treatment will have both chronic benefits in increasing airflow through the airways and will lessen the severity of acute exacerbation of the symptoms of reversible obstructive pulmonary disease.

Application of Treatment

The following illustrations are examples of the invention described herein. It is contemplated that combinations of aspects of specific embodiments or combinations of the specific embodiments themselves are within the scope of this disclosure.

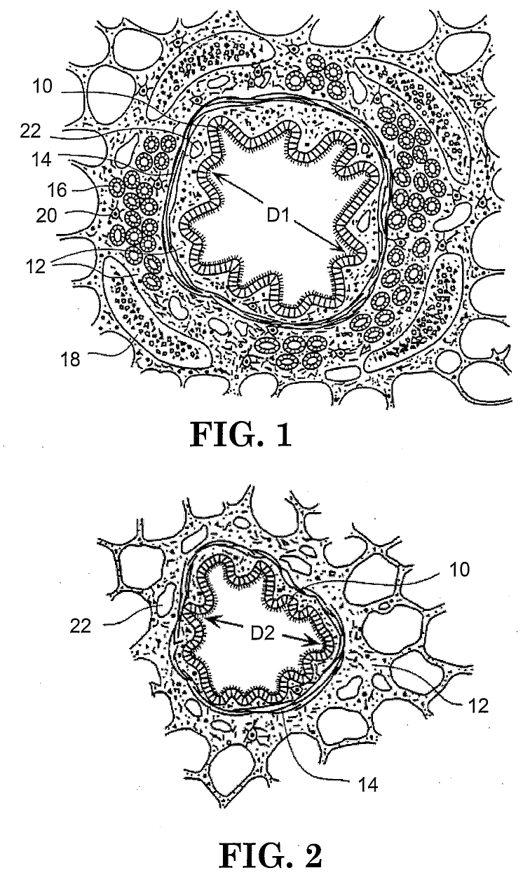

FIGS. 1 and 2 illustrate cross sections of two different airways in a healthy patient. The airway of FIG. 1 is a medium sized bronchus having an airway diameter D1 of about 3 mm. FIG. 2 shows a section through a bronchiole having an airway diameter D2 of about 1.5 mm. Each airway includes a folded inner surface or epithelium 10 surrounded by stroma 12 and smooth muscle tissue 14. The larger airways including the bronchus shown in FIG. 1 also have mucous glands 16 and cartilage 18 surrounding the smooth muscle tissue 14. Nerve fibers 20 and blood vessels 24 also surround the airway.

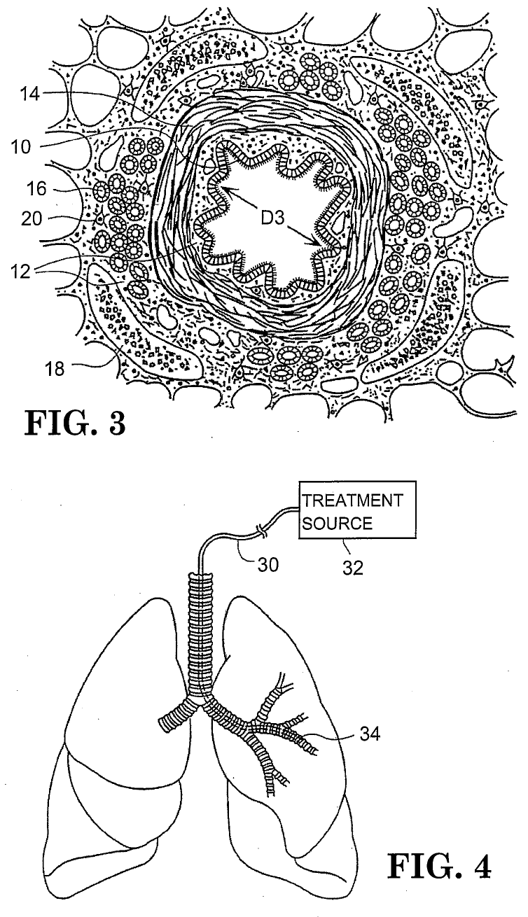

FIG. 3 illustrates the bronchus of FIG. 1 in which the smooth muscle 14 has hypertrophied and increased in thickness causing the airway diameter to be reduced from the diameter D1 to a diameter D3.

FIG. 4 is a schematic side view of the lungs being treated with a treatment device 38 according to the present invention. The treatment device 38 is an elongate member for treating tissue at a treatment site 34 within a lung. Although the invention discusses treatment of tissue at the surface it is also intended that the invention include treatment below an epithelial layer of the lung tissue.

An example of devices for use with the methods of this invention are found in the following U.S. patent application Ser. No. 09/095,323—Methods and Apparatus for Treating Smooth Muscles in the Walls of Body Conduits; Ser. No. 09/349,715—Method of Increasing Gas Exchange of a Lung; and Ser. No. 09/296,040—Devices for Modification of Airways By Transfer of Energy; Ser. No. 09/436,455 Devices for Modification of Airways by Transfer of Energy. The entirety of each of the aforementioned applications is incorporated by reference herein.

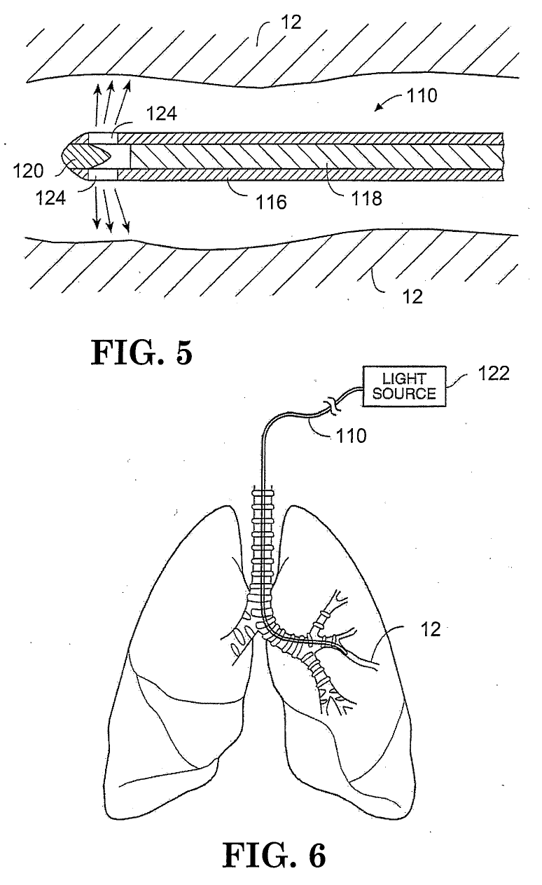

FIGS. 5-12 show another variation of a treatment device for the treatment of airways and other body conduits.

FIG. 5 illustrates an energy delivery device 110 for the delivery of light energy to the walls 12 of a body conduit. The energy delivery device 110 includes an outer catheter or sheath 116 surrounding a light transmitting fiber 118. A light directing member 120 (or a plurality thereof) is positioned at a distal end of the energy delivery device 110 for directing the light to the conduit walls. For example, a plurality of light directing members may redirect light from the fiber in a substantially radial pattern which selectively exposes a length or an inner circumference of the airway wall. Although the present invention will be described in detail with respect to the treatment of airways in the lungs, it should be understood that the present invention may also be used for treatment of other body conduits.

The energy delivery device 110 and method according to the present invention provide a more permanent treatment for asthma than the currently used bronchodilating drugs and drugs for reducing mucus secretion. As discussed above, in asthma patients, the cross sectional diameter of the airways are reduced due to bulking of the smooth muscle surrounding the airways. The energy delivery device 110 of the present invention is used to debulk or reduce the volume of smooth muscle 162 surrounding the airway 160 of an asthma patient and increase the airway diameter for improved air exchange.

The energy delivery device 110 is used to irradiate the smooth muscle surrounding the airways causing the DNA of the smooth muscle cells to become cross linked. The treated smooth muscle cells with cross linked DNA are incapable of replicating. Accordingly, over time, as the smooth muscle cells die, the total thickness of smooth muscle decreases because of the inability of the cells to replicate. The programmed cell death causing a reduction in the volume of tissue is called apoptosis. This treatment does not cause an immediate effect but causes shrinking of the smooth muscle and opening of the airway over time and substantially prevents regrowth. The irradiation by the energy delivery device 110 of the walls of the airway also causes a cross linking of the DNA of the mucus gland cells preventing them from replicating and reducing mucus plugging over time.

As shown in FIG. 6, a variation of the energy delivery device 110 includes an elongate device such as a catheter containing a fiber optic. The energy delivery device 110 is connected by a conventional optical connection to a light source 122. The treatment of an airway with the energy delivery device 110 involves placing a visualization system such as an endoscope or bronchoscope into the airways. The energy delivery device 110 is then inserted through or next to the bronchoscope or endoscope while visualizing the airways. The energy delivery device 110 which has been positioned with a distal end within an airway to be treated is energized so that radiant energy is emitted in a generally radially direction from a distal end of the energy delivery device. The distal end of the energy delivery device 110 is moved through the airway in a uniform painting like motion to expose the a length or an inner circumference of the airway to be treated to the energy. The energy delivery device 110 may be passed along the airway one or more times to achieve adequate treatment. The painting like motion used to expose the length or inner circumference of the airway to the energy may be performed by moving the entire energy delivery device 110 from the proximal end either manually or by motor. Energy delivery may comprise selectively exposing a portion or an entire length or inner circumference of the airway to energy.

The energy used may be coherent or incoherent light in the range of infrared, visible, or ultraviolet. The light source 122 may be any known source, such as a UV laser source. Preferably the light is ultraviolet light having a wavelength of about 240-280 nm or visible light in the red visible range. The intensity of the light may vary depending on the application. The light intensity should be bright enough to penetrate any mucus present in the airway and penetrate the smooth muscle cells and mucus gland cells to cause cross linking of the cell DNA. The light intensity may vary depending on the wavelength used, the application, the thickness of the smooth muscle, and other factors. Alternatively, a beta or gamma radiation source maybe used instead of the light source as described in further detail below with respect to FIGS. 11 and 12.

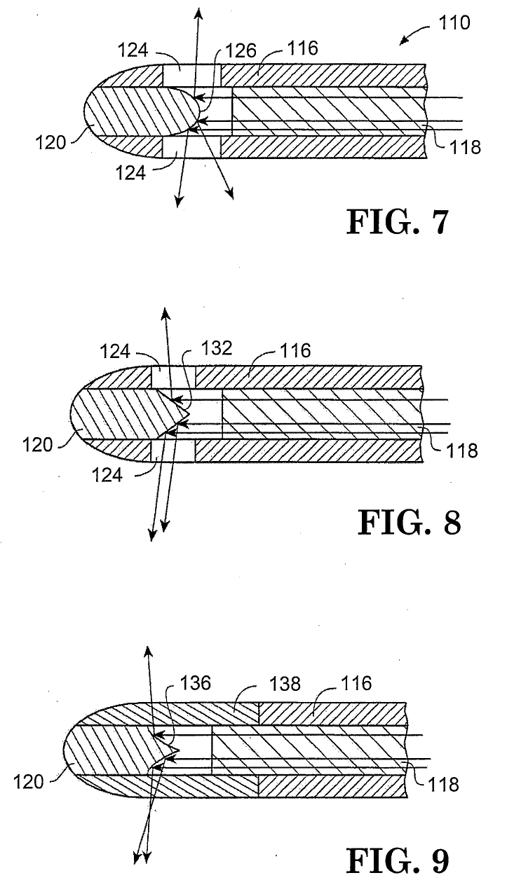

FIGS. 7-10 illustrate different exemplary embodiments of the distal tip of the energy delivery device 110 for irradiating the airway walls. In FIG. 7, the sheath 116 includes a plurality of windows 124 which allow the energy which has been redirected by the light directing member 120 to pass substantially radially out of the sheath. The light directing member 120 is fitted into the distal end of the sheath 116. The light directing member 120 is a parabolic diffusing mirror having a reflective surface 126 which is substantially parabolic in cross section. The light passes from the light source along the light transmitting fiber 118 and is reflected by the reflective surface 126 of the light directing member 120 through the windows 124. The windows 124 are preferably a plurality of energy transmitting sections spaced around the distal end of the sheath. The windows 124 may be open bores extending through the sheath 116. Alternatively, the windows 124 may be formed of a material transparent to the energy being used which allows the energy to pass out of the sheath 116.

FIG. 8 illustrates an alternative embodiment of the energy delivery device 110 in which the light directing member 120 has a conical shaped reflective surface 132. This conical shaped reflective surface may be formed at any desired angle which directs the light transmitted by the light transmitting fiber 118 radially out of the sheath 116. The use of a conical reflective surface 132 creates a light delivery pattern in which the light rays are directed in a generally coherent radial pattern which is at a generally fixed angle with respect to a longitudinal axis of the light delivery device. In contrast, the light delivery device of FIG. 7 with the parabolic reflective surface 126 directs light in a diverging radial pattern which will illuminate a larger area of the airway walls.

FIG. 9 illustrates a further alternative embodiment of the invention in which the light directing member 120 is a substantially conical member including concave reflective surfaces 136. These concave reflective surfaces 136 direct the light which passes in a generally parallel arrangement through the light transmitting fiber 118 out of the sheath 116 in a converging or crossing pattern. In addition, in the embodiment of FIG. 9, the windows have been replaced by a tip 138 of the sheath 116 formed of a material which is transparent to the energy being used.

The light directing members 120 having a reflective surface as illustrated in FIGS. 7-9 may be formed in any of the known manners, such as by coating a molded member with a reflective coating, such as aluminum or silver.

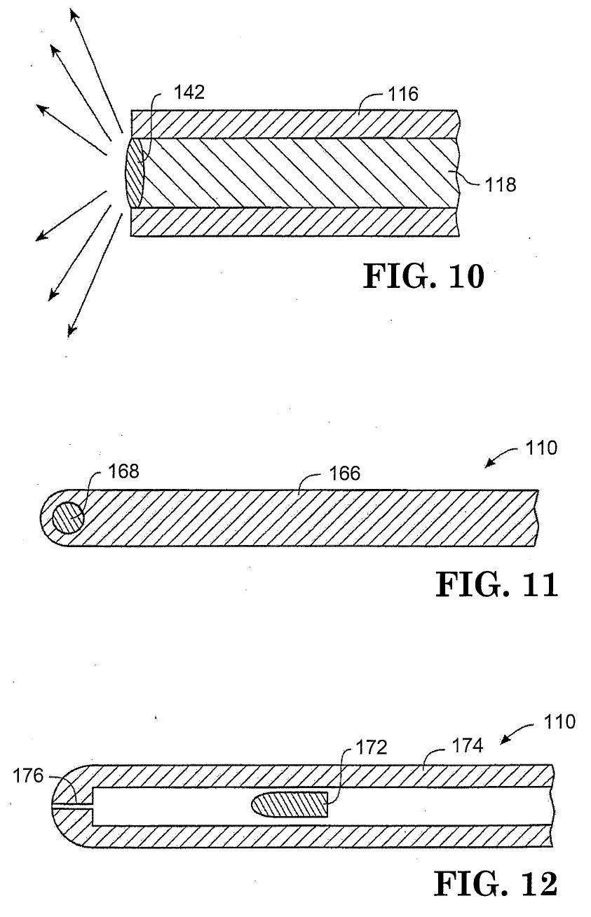

As an alternative to the reflective light directing members of FIGS. 7-9, a diffusing lens 142, such as a Teflon lens, may be positioned at the end of the light transmitting fiber 118 as illustrated schematically in FIG. 10. The diffusing lens 142 may direct the light from the light transmitting fiber 118 in a generally conical pattern as shown in FIG. 10. Alternatively, the diffusing lens 142 may direct the light in a more radially oriented pattern with the light rays being prevented from exiting the lens in a direction substantially parallel with the longitudinal axis of the light transmitting fiber 118 by a reflective or blocking member. In the embodiment of FIG. 10, the sheath 116 surrounding the light transmitting fiber 118 and the diffusing lens 142 may be eliminated entirely and the lens may be affixed directly to the end of the fiber.

According to one alternative embodiment of the invention, the energy delivery device 110 can be used in conjunction with photo-activatable substances such as those known as psoralens. These light activatable compounds, when activated, enhance the ability of light to cross link the DNA in the smooth muscle tissue and mucus glands. The light activatable compound may by injected intravenously. The light delivered by the light delivery device 110 is matched to the absorption spectrum of the chosen light activatable compound such that the light exposure activates the compound. When such light activatable substances are employed, a lower light intensity may be used to achieve cross linking of the DNA than the light intensity required to achieve cross linking without the light activatable compounds.

FIG. 11 illustrates an alternative embodiment of an energy delivery device 110 including an elongate body or shaft 166 having a radiation source 168 positioned at the distal end of the flexible shaft. The radiation source 168 may be any known source of radiation such as a radioactive pellet of iridium. The treatment of a bodily conduit of a patient with the energy delivery device 110 of FIG. 11 is performed by moving the elongate shaft 166 back and forth in the body conduit in a painting like motion to cause a cross linking of the DNA in the smooth muscle surrounding the body conduit.

FIG. 12 illustrates another alternative embodiment of an energy delivery device 110 having a source of radiation such as a radioactive pellet 172 positioned within a cannula 174. According to this embodiment, in addition to moving the cannula itself to achieve a painting action within a body conduit, the pellet 172 may be moved within the cannula 174. Movement of the radioactive pellet 172 may be performed by connecting a syringe to a proximal end of the cannula 174 and injecting or withdrawing fluid through the cannula to move the pellet in a piston like manner. A vent port 176 is provided at the distal end of the cannula 174 to allow fluid to pass into and out of the cannula. In use, the energy delivery device 110 of FIGS. 11 and 12 are preferably delivered to a treatment site within the body through a shielded cannula which prevents radiation from being emitted into surrounding tissue as the device is inserted.

In use, the embodiment of FIG. 12 is inserted to a treatment site such as an airway of the lungs through a radiation shielding cannula. A syringe filled with air is then connected to the proximal end of the cannula 174 and air is injected and withdrawn to move the radioactive pellet within the cannula 174 to expose a desired section of the airway to radiation emitted from the radioactive pellet. Once the treatment has been completed, the cannula 174 and pellet 172 are retracted inside the shielding cannula and the device is withdrawn from the patient.

The cross linking of the smooth muscle and mucus gland DNA according to the present invention will reduce or eliminate the smooth muscle and the secreting glands such as mucus glands from the area of the airway which is treated by preventing the treated cells from replicating. This light treatment provides improved long term relief from asthma symptoms for some asthma sufferers. However, over time, some amount of smooth muscle or mucus gland cells which were not affected by an initial light treatment may regenerate and treatment may have to be repeated after a period of time such as one or more months or years.

Although the present treatment has been described for use in debulking enlarged smooth muscle tissue to open up the airways, it may also be used for eliminating smooth muscle altogether. The elimination of the smooth muscle tissue prevents the hyperreactive airways of an asthma patient from contracting or spasming, completely eliminating this asthma symptom.

The light delivery device 110 may also be used for treatment of other conditions by reducing the volume of smooth muscle tissue surrounding other body conduits. For example, the treatment system may be used for reducing smooth muscle and spasms of the esophagus of patients with achalasia or esophageal spasm, in coronary arteries of patients with Printzmetal's angina variant, for ureteral spasm, for urethral spasm, and irritable bowel disorders.

The treatment of an airway with the treatment device may involve placing a visualization system such as an endoscope or bronchoscope into the airways. The treatment device is then inserted through or next to the bronchoscope or endoscope while visualizing the airways. Alternatively, the visualization system may be built directly into the treatment device using fiber optic imaging and lenses or a CCD and lens arranged at the distal portion of the treatment device. The treatment device may also be positioned using radiographic visualization such as fluoroscopy or other external visualization means. The treatment device which has been positioned with a distal end within an airway to be treated is energized so that energy is applied to the tissue of the airway walls in a desired pattern and intensity. The distal end of the treatment device may be moved through the airway in a uniform painting like motion to expose the entire length of an airway to be treated to the energy. The treatment device may be passed axially along the airway one or more times to achieve adequate treatment. The “painting-like” motion used to exposed the entire length of an airway to the energy may be performed by moving the entire treatment device from the proximal end either manually or by motor. Alternatively, segments, stripes, rings or other treatment patterns may be used.

According to one variation of the invention, the energy is transferred to or from an airway wall in the opening region of the airway, preferably within a length of approximately two times the airway diameter or less, and to wall regions of airways distal to bifurcations and side branches, preferably within a distance of approximately twice the airway diameter or less. The invention may also be used to treat long segments of un-bifurcated airway.

The invention includes a method of advancing a treatment device into a lung and treating the lung with the device to, at least, reduce the ability of the lung to produce at least one symptom of reversible obstructive pulmonary disease. It is contemplated that the treatment may reduce all of the symptoms of reversible obstructive disease. Alternatively, the treatment may be selected to address specific symptoms of the disease. It is also intended that the treatment of the lung may sufficiently reduce the symptoms of reversible obstructive pulmonary disease such that the patient is able to function as those free from the disease. Alternatively, the treatment may be such that the symptoms are reduced to allow the patient to more easily manage the disease. It is also intended that the effects of the treatment may be either long term or short term with repeating treatment necessary to suppress the symptoms.

The methods of the invention described herein may be performed while the lung is experiencing natural symptoms of reversible obstructive pulmonary disease. One such example is where an individual, experiencing an asthma attack, or acute exacerbation of asthma or COPD, undergoes treatment to improve the individual's ability to breath. In such a case, the treatment, called ‘rescue,’ seeks to provide immediate relief for the patient.

The method may also include the steps of locating one or more treatment sites within an airway of the lung, selecting one of the treatment sites from the locating step and treating at least one of the selected treatment sites. As mentioned above, these steps may be, but are not necessarily, performed while the lung is experiencing symptoms of reversible obstructive pulmonary disease.

The invention may further comprise the step of stimulating the lung to produce at least one artificially induced symptom of reversible obstructive pulmonary disease. For example, stimulation of the lung would preferably increase the resistance to airflow within the lung, constrict airways within the lung, inflame/irritate airway tissues, increase edema and/or increase the amount of mucus plugging of the airway. Stimulation of the lung may occur at any point during the procedure or before the procedure. For example, the lung may be stimulated either prior to or after, the step of locating a treatment site. If the lung is stimulated prior to the step of locating a treatment site, the reaction of the stimulated tissue within the lung may be useful in determining which locations are to be selected as treatment sites. The lung tissue or airway tissue within the lung may be stimulated by a variety of methods including but not limited to pharmacological stimulation, (e.g., histamine, methacholine, or other bronchoconstricting agents, etc.), electrical stimulation, mechanical stimulation, or any other stimuli causing obstructive pulmonary symptoms. For example, electrical stimulation may comprise exposing airway tissue to electrical field stimulation. An example of such parameters include 15 VDC, 0.5 ms pulses, 0.5-16 Hz, and 70 VDC, 2-3 ms pulses, 20 HZ.

The locating step described above may be performed using a non-invasive imaging technique, including but not limited to, a bronchogram, magnetic resonance imaging, computed tomography, radiography (e.g., x-ray), and ventilation perfusion scans.

The invention further includes the steps of testing the lung for at least one pre-treatment pulmonary function value prior to treating the lung with the device. After the lung is treated, the lung is re-tested for at least one post-treatment pulmonary function value. Naturally, the two pulmonary function values may be compared to estimate the effect of the treatment. The invention may also include treating additional sites in the lung after the re-testing step to at least reduce the effect of at least one symptom of reversible obstructive pulmonary disease. The invention may also include stimulating the lung to produce at least one artificially induced symptom of reversible obstructive pulmonary disease. As mentioned above, the stimulation of the lung may occur at any point during, or prior to, the procedure. For example, stimulation of the lung may occur prior to the step of testing the lung for pre-treatment pulmonary values. In this case, the values would be determinative of pulmonary function values of a lung experiencing symptoms of reversible obstructive pulmonary disease. Accordingly, the objective is to treat the lung until acceptable pulmonary function values are obtained. One benefit of such a procedure is that the effect of the treatment on the patient is more readily observed as compared to the situation where a patient, having previously been treated, must wait for an attack of reversible obstructive pulmonary disease to determine the efficacy of the treatment.

Pulmonary function values are well known in the art. The following is an example of pulmonary function values that may be used. Other pulmonary function values, or combinations thereof, are intended to be within the scope of this invention. The values include, but are not limited to, FEV (forced expiratory volume), FVC (forced vital capacity), FEF (forced expiratory flow), Vmax (maximum flow), PEFR (peak expiratory flow rate), FRC (functional residual capacity), RV (residual volume), TLC (total lung capacity).

FEV measures the volume of air exhaled over a predetermined period of time by a forced expiration immediately after a full inspiration. FVC measures the total volume of air exhaled immediately after a full inspiration. Forced expiratory flow measures the volume of air exhaled during a FVC divided by the time in seconds. Vmax is the maximum flow measured during FVC. PEFR measures the maximum flow rate during a forced exhale starting from full inspiration. RV is the volume of air remaining in the lungs after a full expiration.

The locating step described above may also comprise identifying treatment sites within the airway being susceptible to a symptom of reversible obstructive pulmonary disease. For example, symptoms may include, but are not limited to, airway inflammation, airway constriction, excessive mucous secretion, or any other asthmatic symptom. Stimulation of the lung to produce symptoms of reversible obstructive pulmonary disease may assist in identifying ideal treatment sites.

As noted above, the method of the present invention may include stimulating the lung to produce at least one artificially induced symptom of reversible obstructive pulmonary disease and further include the step of evaluating the result of stimulation of the lung. For example, the evaluating step may include visually evaluating the effect of the stimulating step on the airway using a bronchoscope with a visualization system or by non-invasive imaging techniques, such as those describe herein. The evaluating step may include measuring pressure changes in the airway before and after the stimulating step. Pressure may be measured globally (e.g., within the entire lung), or locally (e.g., within a specific section of the lung such as an airway or alveolar sac.) Also, the evaluating step may comprise measuring the electrical properties of the tissue before and after the stimulating step. The invention may also include evaluating the results of the stimulating step by combining any of the methods previously mentioned. Also, the invention may further comprise the step of selecting at least one treatment parameter based upon the results of the evaluating step. Such treatment parameters may include, but are not limited to, duration of treatment, intensity of treatment, temperature, amount of tissue treated, depth of treatment, etc.

The method may also include the step of determining the effect of the treatment by visually observing lung, airway or other such tissue for blanching of the tissue. The term “blanching” is intended to include any physical change in tissue that is usually, but not necessarily, accompanied by a change in the color of the tissue. One example of such blanching is where the tissue turns to a whitish color after the treatment of application of energy.

The invention may also include the step of monitoring impedance across a treated area of tissue within the lung. Measuring impedance may be performed in cases of monopolar or bipolar energy delivery devices. Additionally, impedance may be monitored at more than one site within the lungs. The measuring of impedance may be, but is not necessarily, performed by the same electrodes used to deliver the energy treatment to the tissue. Furthermore, the invention includes adjusting the treatment parameters based upon the monitoring of the change in impedance after the treatment step. For example, as the energy treatment affects the properties of the treated tissue, measuring changes in impedance may provide information useful in adjusting treatment parameters to obtain a desired result.

Another aspect of the invention includes advancing a treatment device into the lung and treating lung tissue to at least reduce the ability of the lung to produce at least one symptom of reversible obstructive pulmonary disease and further comprising the step of sub-mucosal sensing of the treatment to the lung tissue. The sub-mucosal sensing may be invasive such as when using a probe equipped to monitor temperature, impedance, and/or blood flow. Or, the sub-mucosal sensing may be non-invasive in such cases as infra-red sensing.

The invention may also include using the treatment device to deposit radioactive substances at select treatment sites within the lung. The radioactive substances, including, but not limited to Iridium (e.g. ¹⁹²Ir.) either treat the lung tissue over time or provide treatment upon being deposited.

The invention also includes scraping epithelial tissue from the wall of an airway within the lung prior to advancing a treatment device into the lung to treat the lung tissue. The removal of the epithelial tissue allows the device to treat the walls of an airway more effectively. The invention further comprises the step of depositing a substance on the scraped wall of the airway after the device treats the airway wall. The substance may include epithelial tissue, collagen, growth factors, or any other bio-compatible tissue or substance, which promotes healing, prevent infection, and/or assists in the clearing of mucus. Alternatively, the treatment may comprise the act of scraping epithelial tissue to induce yield the desired response.

The invention includes using the treating device to pre-treat the lung to at least reduce the ability of the lung to produce at least one symptom of reversible obstructive pulmonary disease prior to the treating step. At least one of the parameters of the pre-treating step may differ than one of the parameters of the treating step. Such parameters may include time, temperature, amount of tissue over which treatment is applied, amount of energy applied, depth of treatment, etc.

The invention may also include advancing the treatment device into the lung and treating the lung tissue in separate stages. One of the benefits of dividing the treating step into separate stages is that the healing load of the patient is lessened. Dividing of the treating step may be accomplished by treating different regions of the lung at different times. Or, the total number of treatment sites may be divided into a plurality of groups of treatment sites, where each group of treatment sites is treated at a different time. The amount of time between treatments may be chosen such that the healing load placed on the lungs is minimized.

The invention may also include advancing a treatment device into the lung, treating the lung with the device and sensing movement of the lung to reposition the treatment device in response to the movement. This sensing step accounts for the tidal motion of the lung during breathing cycles or other movement. Taking into account the tidal motion allows improved accuracy in repositioning of the device at a desired target.

The invention may also include the additional step of reducing or stabilizing the temperature of lung tissue near to a treatment site. This may be accomplished for example, by injecting a cold fluid into lung parenchyma or into the airway being treated, where the airway is proximal, distal, or circumferentially adjacent to the treatment site. The fluid may be sterile normal saline, or any other bio-compatible fluid. The fluid may be injected into treatment regions within the lung while other regions of the lung normally ventilated by gas. Or, the fluid may be oxygenated to eliminate the need for alternate ventilation of the lung. Upon achieving the desired reduction or stabilization of temperature the fluid may be removed from the lungs. In the case where a gas is used to reduce temperature, the gas may be removed from the lung or allowed to be naturally exhaled. One benefit of reducing or stabilizing the temperature of the lung may be to prevent excessive destruction of the tissue, or to prevent destruction of certain types of tissue such as the epithelium, or to reduce the systemic healing load upon the patient's lung.

Also contemplated as within the scope of the invention is the additional step of providing therapy to further reduce the effects of reversible obstructive pulmonary disease or which aids the healing process after such treatment. Some examples of therapy include, drug therapy, exercise therapy, and respiratory therapy. The invention further includes providing education on reversible obstructive pulmonary disease management techniques to further reduce the effects of the disease. For example, such techniques may be instruction on lifestyle changes, self-monitoring techniques to assess the state of the disease, and/or medication compliance education.

There may be occurrences where it is necessary to reverse the effects of the treatment described herein. Accordingly, the invention further includes a method for reversing a treatment to reduce the ability of the lung to produce at least one symptom of reversible obstructive pulmonary disease comprising the step of stimulating re-growth of smooth muscle tissue. The re-stimulation of the muscle may be accomplished by the use of electro-stimulation, exercising of the muscle and/or drug therapy.

The invention further includes methods of evaluating individuals having reversible obstructive pulmonary disease, or a symptom thereof, as a candidate for a procedure to reduce the ability of the individual's lung to produce at least one symptom of reversible obstructive pulmonary disease. The method comprises the steps of assessing the pulmonary condition of the individual, comparing the pulmonary condition to a corresponding pre-determined state, and evaluate the individual as a candidate based upon the comparison.

In assessing the pulmonary condition, the method may comprise the steps of performing pulmonary function tests on the individual to obtain a pulmonary function value which is compared to a predetermined value. Examples of pre-determined values are found above.

The method of evaluating may further include the step of determining how the individual's tissue will react to treatment allowing the treatment to be tailored to the expected tissue response.

The method of evaluating may further comprises the step of pulmonary function testing using a gas, a mixture of gases, or a composition of several mixtures of gases to ventilate the lung. The difference in properties of the gases may aid in the pulmonary function testing. For example, comparison of one or more pulmonary function test values that are obtained with the patient breathing gas mixtures of varying densities may help to diagnose lung function. Examples of such mixtures include air, at standard atmospheric conditions, and a mixture of helium and oxygen. Additional examples of pulmonary testing include tests that measure capability and evenness of ventilation given diffusion of special gas mixtures. Other examples of gases used in the described tests, include but are not limited to, nitrogen, carbon monoxide, carbon dioxide, and a range of inert gases.

The invention may also comprise the step of stimulating the lung to produce at least one artificially induced symptom of reversible obstructive pulmonary disease. Stimulating the symptoms of the disease in an individual allows the individual to be evaluated as the individual experiences the symptoms thereby allowing appropriate adjustment of the treatment.

The method of evaluating may also comprise the step of obtaining clinical information from the individual and accounting for the clinical information for treatment.

The method may further comprise the selection of a patient for treatment based upon a classification of the subtype of the patient's disease. For example, in asthma there are a number of ways to classify the disease state. One such method is the assessment of the severity of the disease. An example of a classification scheme by severity is found in the NHLBI Expert Panel 2 Guidelines for the Diagnosis and Treatment of Asthma. Another selection method may include selecting a patient by the type of trigger that induces the exacerbation. Such triggers may be classified further by comparing allergic versus non-allergic triggers. For instance, an exercise induced bronchospasm (EIB) is an example of a non-allergenic trigger. The allergic sub-type may be further classified according to specific triggers (e.g., dust mites, animal dander, etc.). Another classification of the allergic sub-type may be according to characteristic features of the immune system response such as levels of IgE (a class of antibodies that function in allergic reactions, also called immunoglobulin). Yet another classification of allergic sub-types may be according to the expression of genes controlling certain interleukins (e.g., IL-4, IL-5, etc.) which have been shown to play a key role in certain types of asthma.

The invention further comprises methods to determine the completion of the procedure and the effectiveness of the reduction in the lung's ability to produce at least one symptom of reversible obstructive pulmonary disease. This variation of the invention comprises assessing the pulmonary condition of the individual, comparing the pulmonary condition to a corresponding predetermined state, and evaluating the effectiveness of the procedure based on the comparison. The invention may also comprise the steps of performing pulmonary function tests on the individual to obtain at least one pulmonary function value, treating the lung to at least reduce the ability of the lung to produce at least one symptom of reversible obstructive pulmonary disease, performing a post-procedure pulmonary function tests on the individual to obtain at least one post pulmonary function value and comparing the two values.

This variation of the invention comprises obtaining clinical information, evaluating the clinical information with the results of the test to determine the effectiveness of the procedure. Furthermore, the variation may include stimulating the lung to produce a symptom of reversible obstructive pulmonary disease, assessing the pulmonary condition of the patient, then repeating the stimulation before the post-procedure pulmonary therapy. These steps allow comparison of the lung function when it is experiencing symptoms of reversible obstructive pulmonary disease, before and after the treatment, thereby allowing for an assessment of the improved efficiency of the lung during an attack of the disease.

The invention herein is described by examples and a desired way of practicing the invention is described. However, the invention as claimed herein is not limited to that specific description in any manner. Equivalence to the description as hereinafter claimed is considered to be within the scope of protection of this patent.

We claim as our invention:

1. An energy emitting device for treatment of a hyper-reactive airway in a human lung, the device comprising:
an elongate body having a proximal end and a distal end configured to be inserted into a human lung; and
a source of energy for emitting energy from the elongate body at a wavelength and intensity which, when applied to a wall of a hyper-reactive airway causes a change in smooth muscle tissue so as to prevent the airway from contracting.

2. The device of claim 1, wherein the source of energy comprises:
a light transmitting fiber extending from the proximal end to the distal end of the elongate body; and
a light directing member positioned at a distal end of the elongate body for diffusing or redirecting the light from the light transmitting fiber in a substantially radial pattern from the distal end of the elongate body.

3. The device of claim 2, wherein the wavelength is in a range from about 240 nm to about 280 nm.

4. The device of claim 2, wherein the wavelength is in a red visible range.

5. The device of claim 2, wherein the light directing member includes a substantially conical reflective surface which redirects light from the light transmitting fiber in a direction away from a longitudinal axis of the fiber.

6. The device of claim 5, wherein the reflective surface comprises a concave, substantially planar, or substantially parabolic cross section.

7. The device of claim 2, wherein the light directing member includes a diffusing lens which directs light from the transmitting fiber in a direction away from a longitudinal axis of the fiber.

8. The device of claim 2, wherein the light transmitting fiber is surrounded by a sheath for delivery to the airway.

9. The device of claim 8, wherein the sheath includes a distal section which is transparent to the energy emitted by the light source.

10. The device of claim 8, wherein the sheath includes a distal section comprising a plurality of windows that are transparent to the energy emitted by the light source to allow the light which has been redirected by the light directing member to exit the sheath.

11. The device of claim 1, wherein the source of energy comprises:
a light transmitting fiber extending from the proximal end to the distal end of the elongate body; and
a plurality of light directing members positioned at a distal end of the elongate body for redirecting the light from the light transmitting fiber in a substantially radial pattern which selectively exposes a length or an inner circumference of the airway wall.

12. The device of claim 1, wherein the source of energy emits energy at the wavelength and intensity which, when applied to the airway crosslinks DNA in smooth muscle cells surrounding the airway and prevents the smooth muscle cells from replicating.

13. The device of claim 1, wherein the source of energy comprises a radioactive pellet positioned at the distal end of the elongate body.

14. The device of claim 1, wherein the source of energy comprises a radioactive pellet which is movable longitudinally within the elongate body.

15. An asthma treatment system for emitting light to a wall of an airway in a human lung, the system comprising:
an elongate body having a proximal end and a distal end configured to be inserted into a human lung; and
a laser source capable of emitting light from the distal end of the elongate body and at an intensity which, when applied to a wall of an airway reduces smooth muscle tissue so as to treat asthma.

16. The system of claim 15, wherein the elongate body further comprises a light directing member located at the distal end of the elongate member and coupled to the laser source, wherein the light directing member comprises a reflective surface that directs the light in a substantially radial pattern from the distal end of the elongate body.

17. The system of claim 16, wherein the reflective surface is parabolic so that the radially directed light diverges.

18. The system of claim 16, wherein the reflective surface is concave so that the radially directed light converges.

19. The system of claim 16, wherein the reflective surface diffuses light from the distal end of the elongate body.

20. The system of claim 16, further comprising a sheath having a distal section comprising a plurality of windows that are transparent to the energy emitted by the laser source to allow the light which has been redirected by the light directing member to exit the sheath.

21. An energy emitting device for treatment of an airway in a human lung, the device comprising:
an elongate body having a proximal end and a distal end configured to be inserted into a human lung; and
a source of energy for emitting energy from the elongate body at a wavelength and intensity which, when applied to a wall of an airway causes a change in smooth muscle tissue so as to prevent the smooth muscle tissue from replicating.
